(19) United States
(12) Patent Application Publication
(10) Pub. No.: US 2013/0338649 A1
(43) Pub. Date: Dec. 19, 2013

(54) OPHTHALMIC LASER SURGICAL APPARATUS

(71) Applicant: NIDEK CO., LTD., Gamagori-shi (JP)

(72) Inventors: Masaaki HANEBUCHI, Gamagori-shi (JP); Masaki TANAKA, Gamagori-shi (JP)

(73) Assignee: NIDEK CO., LTD., Gamagori-shi (JP)

(21) Appl. No.: 13/907,324

(22) Filed: May 31, 2013

(30) Foreign Application Priority Data
Jun. 2, 2012 (JP) 2012-126626

(51) Int. Cl. A61F 9/008

(52) U.S. Cl. CPC A61F 9/0084
USPC 606/4

(57) ABSTRACT

An ophthalmic laser surgical apparatus includes: a laser irradiation optical system configured to irradiate a patient's eye with surgical laser; a fixation unit configured to fix the patient's eye on the laser irradiation optical system; an interface unit including an optical member configured to cover at least the cornea of the patient's eye; a tomography unit configured to capture a tomographic image of the anterior segment of the patient's eye; and a monitoring unit configured to monitor a position of the fixation unit and/or the interface unit with respect to the patient's eye based on the tomographic image captured by the tomography unit.

### CROSS-REFERENCE TO RELATED APPLICATION

This application is based on Japanese Patent Application No. 2012-126626 filed with the Japan Patent Office on Jun. 2, 2012, the entire content of which is hereby incorporated by reference.

### BACKGROUND

1. Technical Field

The present disclosure relates to an ophthalmic laser surgical apparatus for irradiating a patient's eye with laser to effect incision of the tissue or the like.

2. Related Art

In recent years, a technique for incising (breaking up) a tissue such as the cornea or crystalline lens of a patient's eye (an eye to be operated) using a femtosecond order pulse laser (an ultra-short pulse laser beam) has been proposed (see, for example, JP-T-2004-531344 (Patent Document 1)). Such an apparatus changes the location of laser spots three dimensionally to simultaneously allow the laser spots to be seamlessly connected to one another, thereby effecting incision of the eye tissue or the like. The apparatus includes a member for preventing the eye from moving during laser irradiation. The member includes an interface unit (contact unit) as well as a suction unit for suctioning the eye. In order to accurately guide a laser spot (to effect the positioning of an optical system), for example, the interface unit applanates the cornea.

### SUMMARY

An ophthalmic laser surgical apparatus includes: a laser irradiation optical system configured to irradiate a patient's eye with surgical laser; a fixation unit configured to fix the patient's eye on the laser irradiation optical system; an interface unit including an optical member configured to cover at least the cornea of the patient's eye; a tomography unit configured to capture a tomographic image of the anterior segment of the patient's eye; and a monitoring unit configured to monitor a position of the fixation unit and/or the interface unit with respect to the patient's eye based on the tomographic image captured by the tomography unit.

### BRIEF DESCRIPTION OF DRAWINGS

FIG. 1 is a diagram illustrating an ophthalmic laser surgical apparatus according to an embodiment;

FIG. 2 is a cross-sectional view of an eye fixation/interface unit;

FIG. 3 is a cross-sectional view of the eye fixation/interface unit where the interface unit is located at an uppermost position;

FIG. 4 is a diagram illustrating a monitor display; and

FIG. 5 is a block diagram illustrating an observation and photographing unit.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

If the eye is fixed by an apparatus described in Patent Document 1, for example, an operator presses a contact glass against the cornea. Consequently, the operator optically fixes the eye with respect to the contact glass. The apparatus of Patent Document 1 makes the operator difficult to grasp a positional relationship between a fixation unit such as a contact glass and a patient's eye.

An object of the present disclosure is to provide an ophthalmic laser surgical apparatus that can achieve suitable eye fixation dependent on a patient's eye.

An ophthalmic laser surgical apparatus includes: a laser irradiation optical system configured to irradiate a patient's eye with surgical laser; a fixation unit configured to fix the patient's eye on the laser irradiation optical system; an interface unit including an optical member configured to cover at least the cornea of the patient's eye; a tomography unit configured to capture a tomographic image of the anterior segment of the patient's eye; and a monitoring unit configured to monitor a position of the fixation unit and/or the interface unit with respect to the patient's eye based on the tomographic image captured by the tomography unit.

According to the ophthalmic laser surgical apparatus, eye fixation can be suitably performed dependent on a patient's eye.

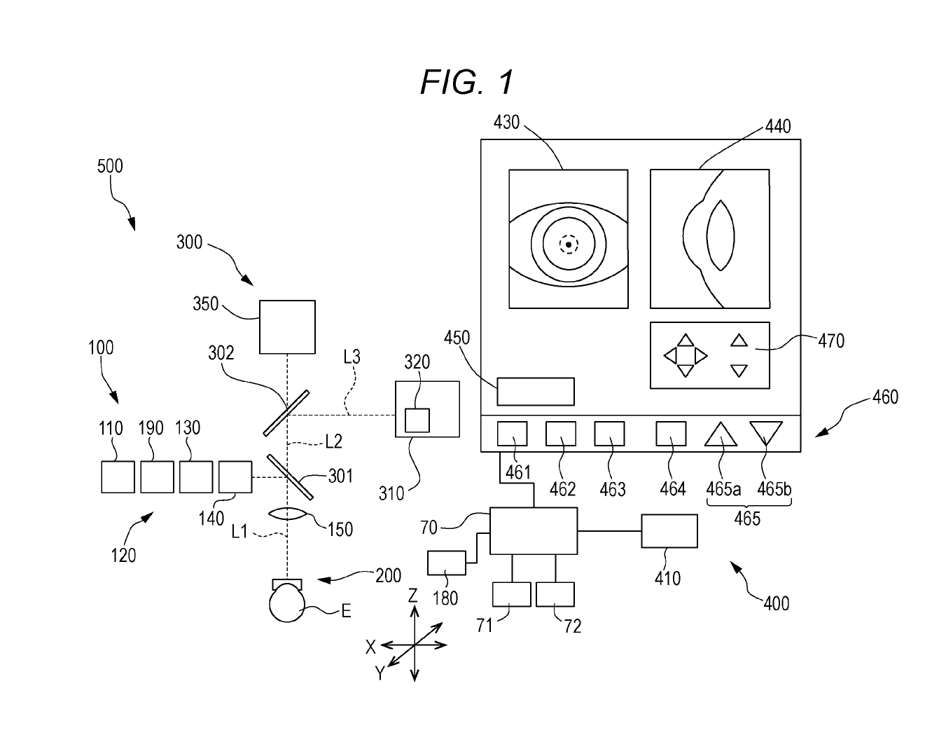

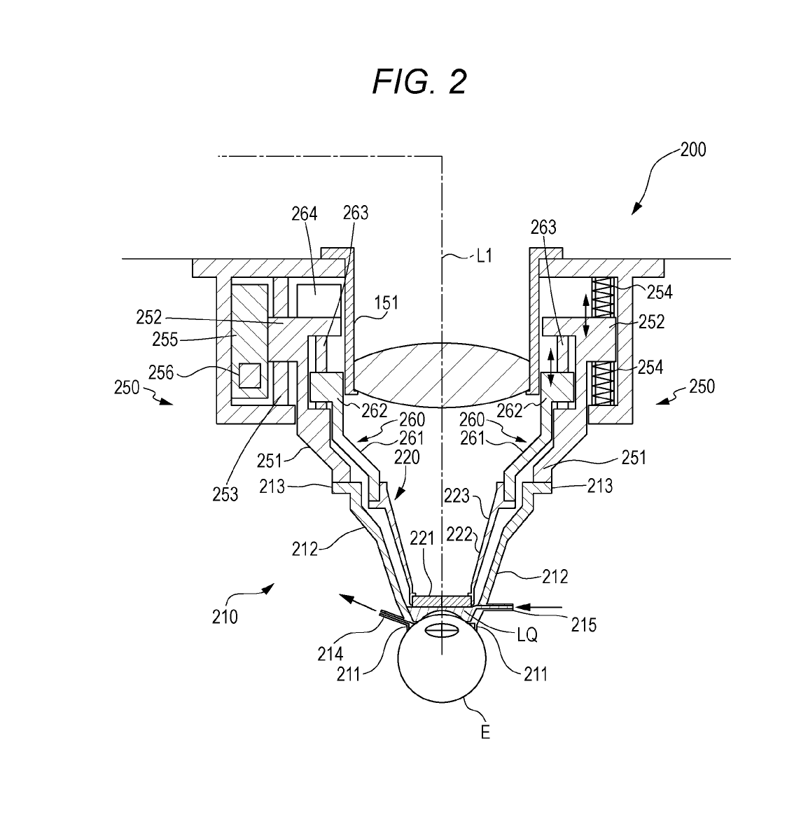

Hereinafter, an ophthalmic laser surgical apparatus (the present apparatus) according to an embodiment of the present disclosure will be described with reference to the drawings. FIG. 1 is a schematic diagram illustrating the configuration of the present apparatus. FIG. 2 is a diagram illustrating the configuration of a fixation unit of the present apparatus. In the embodiment, the axial direction (depth direction) of a patient's eye E is defined as a Z direction, the horizontal direction as an X direction, and the vertical direction as a Y direction.

<Entire Configuration of the Present Apparatus>

The configuration of the present apparatus will be schematically described. In the present apparatus, an eye tissue (a crystalline lens LE) of the patient's eye (the eye to be operated) E is irradiated with surgical laser (a laser beam). The laser incises and breaks up the crystalline lens.

An ophthalmic laser treatment apparatus 500 mainly includes a laser irradiation unit (main body unit) 100, an eye fixation/interface unit 200, an observation and photographing unit 300, an operating unit 400, and a control unit 70.

The laser irradiation unit 100 irradiates the patient's eye E with pulse laser. The eye fixation/interface unit 200 fixes and holds the patient's eye E to the laser irradiation unit 100. The observation and photographing unit 300 captures a front image of the anterior segment of the patient's eye E and a tomographic image of the anterior segment. The operating unit 400 is an operating unit for an operator to operate the present apparatus 500. The control unit 70 oversees and excises control over the entire present apparatus.

The observation and photographing unit 300 includes an optical coherence tomography unit (abbreviated to OCT (Optical Coherence Tomography) unit) 310, and a front observing unit 350. The OCT unit 310 captures (obtains) a tomographic image of the patient's eye E. The front observing unit 350 photographs an anterior segment image of the patient's eye E.

<Laser Irradiation Unit>

The laser irradiation unit 100 includes a laser light source unit 110, a laser irradiation optical system 120, and a position detecting unit 190. The laser light source unit 110 emits surgical pulse laser (a laser beam). The laser irradiation optical system 120 includes an optical member for guiding the laser (laser light). The position detecting unit 190 detects the absolute position of the patient's eye. The laser irradiation optical system 120 is built in a main body of the present apparatus 500.

The laser irradiation optical system (laser delivery) 120 includes a beam expander unit 130, a scanning unit 140, and an objective lens 150. The beam expander unit 130 moves a laser spot in any direction. The scanning unit 140 moves a laser spot in the X and Y directions. The objective lens 150 is a condensing optical system (image forming optical system) that condenses the laser as a laser spot at a target position. The laser irradiation optical system 120 further includes various optical members for guiding the laser.

The laser light source unit 110 is a laser light source that emits a pulse laser. Such a laser light source generates plasma (causes a breakdown), for example, at a focal point (the position of a laser spot).

Plasma is generated at the focal point (the position of a laser spot). A breakdown (optical breakdown) is caused by the plasma at the target position (spot position). An eye tissue at the target position is then mechanically destroyed. The laser spots are connected to incise and brake up the eye tissue (for example, the crystalline lens). For example, a device that emits pulse laser with pulse widths of one femtosecond to 10 nanoseconds is used as the laser light source unit 110. The laser light source unit 110 according to the embodiment emits, for example, a pulse laser in the ultraviolet range, the pulse laser having a pulse width of 10 picoseconds, a wavelength width of ±10 nm, and a center wavelength of 450 nm. Moreover, laser that can be emitted by the laser light source unit 110 has a power enough to cause a breakdown with a laser spot of 1 to 15 μm in spot size. The laser light source unit (laser light source) 110 may be a device that emits pulse laser in infrared range, the pulse laser having a pulse width of 500 femtoseconds and a wave center wavelength of 1040 nm (a pulse width of ±10 nm).

The beam expander unit 130 (hereinafter simply referred to as the expander) includes a plurality of optical elements. The expander 130 changes divergence of a beam of the pulse laser passing through itself. Consequently, the expander 130 can move a laser spot in the Z direction (on an optical axis L1).

In the expander 130 of the embodiment, a lens having a negative refractive power and a lens having a positive refractive power are arranged from the upstream side (from the laser light source unit 110's side). The upstream lens is configured to move along an optical axis. Consequently, the divergence (divergence angle, convergence angle, or the like) of the beam that has passed through the expander 130 can be changed. The condensing position of the laser spot is shifted in the Z direction depending on the divergence of the pulse laser incident on the objective lens 150.

The scanning unit (optical scanner unit) 140 includes a galvanometer mirror for moving the laser in the X direction and a galvanometer minor for moving the laser in the Y direction.

The scanning unit 140 is configured to allow the laser to scan in the X and Y directions. The scanning unit 140 may include, for example, a polygon mirror for scanning in the X direction and a galvanometer mirror for scanning in the Y direction. Moreover, the scanning unit 140 may be configured to use a resonant mirror in both the X and Y directions. Moreover, the scanning unit 140 may be configured to rotate two prisms independently of each other.

In this manner, in the present apparatus 500, the laser spot is moved three-dimensionally (in the X, Y and Z directions) by the expander 130 and the scanning unit 140 in an eye tissue (in a target) of the patient's eye E. The expander 130 and the scanning unit 140 are included in a moving optical system. The expander 130 is arranged upstream of the scanning unit 140. Consequently, the laser can be prevented from passing through the expander 130 after being directed to the X and Y directions. Hence, the effective diameter and size of the optical member of the expander 130 can be reduced. Furthermore, a lens 131 can be made smaller. Consequently, the laser spot can move faster in the Z direction.

A beam combiner (beam splitter) 301 is arranged between the scanning unit 140 and the objective lens 150. The beam combiner 301 is coaxial with a laser optical axis and an observation and photographing optical axis. The combiner 301 has a characteristic that reflects the pulse laser while transmitting the illuminating light of the observation and photographing unit. The objective lens 150 is a lens arranged fixedly to the main body of the apparatus. As illustrated in FIG. 2, the objective lens 150 is held by a lens holder 151 fixed to the main body of the apparatus. The objective lens 150 allows the laser to form an image on a target to give a minute laser spot of approximately 1 to 15 μm.

Although an illustration is omitted, the laser irradiation unit 100 has an aiming light source. The aiming light source emits aiming light (aiming light) to be used for the operator to check a laser irradiation position.

<Position Detecting Unit>

Some members of the position detecting unit 190 also serve as those of the laser irradiation optical system 120. The position detecting unit 190 includes a confocal aperture plate and a light receiving device. An aperture of the confocal aperture plate is arranged at a position substantially conjugated with the position of the laser spot. Consequently, the light receiving device can detect reflected light at the laser spot position (reflected light at a tissue).

Information on the absolute position of the laser spot is obtained from position information of the expander 130 and the optical element of the scanning unit 140. For example, the reflected light at the laser spot position is received by the light receiving device and the intensity of the reflected light is obtained. Thus, information on the absolute position of the laser spot (target) is obtained. Upon detection of the absolute position, the output of the laser light source unit 110 is reduced by an attenuator or the like. Consequently, the occurrence of a breakdown at the laser spot position is prevented.

The position detecting unit 190 may be configured to detect the absolute position of a feature portion of the patient's eye, or may be configured to obtain control information of the laser irradiation optical system 120 with a position detection operation.

<Eye Fixation/interface Unit Unit>

The eye fixation/interface unit 200 fixes (holds) the patient's eye E to the laser irradiation optical system 120 (the main body of the apparatus). Furthermore, the eye fixation/interface unit 200 is responsible for guiding the laser to a tissue of the patient's eye E. The eye fixation/interface unit 200 is arranged in the vicinity of the end of the laser irradiation optical system 120 (in the vicinity of the objective lens 150). The objective lens 150 is held by the lens holder 151 arranged fixedly to the main body of the apparatus. The eye fixation/interface unit 200 mainly includes a suction ring (a suction unit and a fixation unit) 210, a first holding unit (suction ring holding unit) 250, an interface unit 220, and a second holding unit 260.

The suction ring 210 fixes the patient's eye E by suction. The first holding unit 250 holds the suction ring 210 to the main body of the apparatus. The interface unit 220 covers the cornea of the patient's eye E and guides the laser to the eye tissue. The second holding unit 260 holds the interface unit 220 to the main body of the apparatus.

The suction ring 210 includes a ring 211, a support part 212, a suction-purpose pipe 214, and a pipe 215. The ring 211 is a contact part that comes into contact with the sclera of the patient's eye E. The support part 212 supports the ring 211. The suction-purpose pipe 214 is a passage (venthole) to apply suction pressure to the ring 211. The pipe 215 is a passage (through hole) formed by penetrating the support part 212. The pipe 215 is a pipe for liquid supply/drain to supply or drain liquid. The suction ring 210 has a conical shape. In other words, the suction ring 210 has a tapered part from a proximal part 213 formed at a proximal end of the support part 212 toward the ring 211. The suction ring 210 transfers (adds) suction pressure added by a suction pump (illustration omitted) to the ring 211 through the suction-purpose pipe 214.

The ring 211 has an opening along the curved surface of the sclera. The opening of the ring 211 is a shaped so that it can surround the ring (the vicinity of the cornea) of the eye, thereby having a ring shape that corresponds to the shape of the ring 211. Consequently, the eye is uniformly suctioned to the ring 211. The support part 212 has a wall shape that surrounds the ring 211 (the eye) such that liquid can be filled inside the support part 212 in a state where the eye is suctioned to the ring 211.

Moreover, the suction ring 210 has a space to accommodate the interface unit 220 without contact (interference), the space being formed inside the support part 212. The proximal part 213 of the support part 212 has a shape (fitting shape) that the support part 212 is detachable from the first holding unit 250 (the details are described below).

A suction tube from the suction pump provided to the main body of the apparatus (or of a separate unit) is connected to the suction-purpose pipe 214. Negative pressure is generated inside the suction tube, the suction-purpose pipe 214, and the ring 211 by the suction pressure generated by the operation of the suction pump. Consequently, the sclera of the patient's eye E is suctioned to the opening of the ring 211. An end (opening) of the pipe 215 is formed in an inner wall of the support part 212.

The pipe 215 is connected through a tube or the like to an irrigation suction unit whose illustration is omitted. Before laser irradiation, liquid is supplied into the suction ring 210 by the operation of the irrigation suction unit. After surgery, the liquid inside the suction ring 210 is drained. The pipe 215 may be configured not to drain the liquid. In this case, the operator removes the suction ring 210 from the patient's eye E to discard the liquid.

The material of the suction ring 210 contains, for example, a resin or metal that is biocompatible. The suction ring 210 is a disposable type to be disposed of after a single use. Therefore, it is preferred that the suction ring 210 be configured to be detachable from the first holding unit 250.

The first holding unit 250 is attached to the main body of the apparatus. The first holding unit 250 includes a holding part (fitting part) 251, a rail (shaft) 253, elastic members 254, a regulation part 255, and a sensor 256. The holding part (fitting part) 251 fits to the proximal part 213. The rail (shaft) 253 guides the movement in the Z direction of a proximal part 252 formed at a proximal end of the holding part 251. The rail 253 is a guide mechanism provided to the present apparatus 500. The elastic members 254 are respectively arranged above and beneath the proximal part 252. The regulation part 255 regulates the movement of the holding part 251. The sensor 256 detects the position of the holding part 251 in the Z direction.

The holding part 251 has a ring shape with a diameter larger than that of the objective lens 150. The holding part 251 (the distal end thereof) holds the support part 212 detachably. Therefore, the holding part 251 (the distal end thereof) has a structure that the holding part 251 (the distal end thereof) and the proximal part 213 of the support part 212 are fitted to each other. The holding part 251 is responsible for holding the suction ring 212 as well as effecting the positioning of the holding part 251 (the distal end thereof). For example, a groove (ring-shaped groove) may be formed on the holding part 251. Furthermore, a protrusion (ring-shaped protrusion) may be formed on the proximal part 213. In this case, the proximal part 213 is pressed against the holding part 251 to fit the protrusion into the groove. Consequently, the suction ring 210 is held by the holding part 251.

Alternatively, both the holding part 251 and the proximal part 213 may respectively have screw threads which are screwed together. In this case, the proximal part 213 is screwed (fitted to) the holding part 251 by the rotation of the suction ring 210. Moreover, the holding part 251 may be configured to detachably hold the proximal part 213 by magnetic force, suction, or the like.

The proximal part 252 of the holding part 251 forms a flange of the holding part 251. The proximal part 252 of the holding part 251 has a ring shape. A hole (reference numeral omitted), into which the rail 253 can fit, is formed in the proximal part 252 and extends in the Z direction. The rail 253 is fixedly arranged to the main body of the apparatus. The rail 253 is arranged along the Z direction (corresponding to the up-down direction) to set the movement direction of the proximal part 252 to the Z direction.

The elastic members 254 are respectively arranged on upper and lower sides of the proximal part 252. The proximal part 252 of the holding part 251 and the rail 253 are a moving mechanism to enable the first holding unit 250 and the suction ring 210 to move in the Z direction.

The first holding unit 250 is used as a moving unit (alignment unit) to move the suction ring 210. The elastic members 254 are arranged along the rail 253 in the Z direction to bias the proximal part 252 in the Z direction. The elastic members 254 have biasing force (restoring force). The elastic members 254 may be, for example, a spring or rubber. The elastic member 254 located on the upper side of the proximal part 252 is fixed at an upper end thereof, and is in contact with (is fixed to) the proximal part 252 at a lower end thereof. The elastic member 254 located on the lower side of the proximal part 252 is in contact with (is fixed to) the proximal part 252 on an upper side thereof, and is fixed at a lower end thereof. The elastic member 254 located on the lower side of the proximal part 252 biases the proximal part 252 upward. The elastic member 254 located on the upper side of the proximal part 252 biases the proximal part 252 downward. The proximal part 252 (the holding part 251 and the like) is balanced and located on the rail 253 by the elastic members 254.

If an excessive external force is applied to the proximal part 252 in a state where the proximal part 252 is biased by the elastic members 254, the proximal part 252 (the holding part 251), which has received the external force, escapes against the biasing force. For example, if the suction ring 210 moves downward in a state where the patient's eye E is being suctioned to the suction ring 210, the patient's eye E is pressed by the suction ring 210. If the pressure that the patient's eye E receives exceeds the biasing force of the elastic members 254, the proximal part 252 receives an upward force. Consequently, the suction ring 210, the holding part 251, and the proximal part 252 move (escape) upward. Consequently, the patient's eye E hardly receives more than a predetermined pressing force (the force on the patient's eye E is cushioned). In this manner, the first holding unit (moving unit) 250 has a force absorbing mechanism in the Z direction.

The regulation part 255 is responsible for fixing (locking) the position of the proximal part 252 in the Z direction. In the embodiment, the regulation part 255 has a mechanism for holding the proximal part 252. The regulation part 255 is configured to fix the position of the proximal part 252 at any position in the Z direction based on an instruction signal. The regulation part 255 is connected to the control unit 70. The regulation part 255 fixes (regulates) the proximal part 252 based on a signal from the control unit 70.

The sensor 256 is responsible for detecting the position of the holding part 251 in the Z direction. For example, the sensor 256 is a potentiometer that detects the position of the holding part 251. The sensor 256 is connected to the control unit 70. The sensor 256 transmits position detection information to the control unit 70. The control unit 70 fixes the position of the proximal part 252 by transmitting an instruction signal to the regulation part 255 based on the detection signal from the sensor 256. The regulation part 255 fixes the proximal part 252, for example, on a condition that the position of the patient's eye E with respect to the laser irradiation optical system 120 (the objective lens 150) is within an optically appropriate area for laser irradiation (an alignment area). The sizes of the suction ring 210, the holding part 251, and the proximal part 252 are known in terms of design. Therefore, the control unit 70, which has received the detection signal of the sensor 256, can grasp the position of the ring 211 with respect to the objective lens 150.

The sensor 256 may be a photo sensor. For example, a light shielding plate may be attached to the proximal part 252. The photo sensor and the light shielding plate constitute a photo interrupter. In this case, the photo sensor is configured, for example, to transmit the detection signal to the control unit 70 when the proximal part 252 is within a predetermined area.

The interface unit 220 is arranged close to the cornea of the patient's eye E. The interface unit 220 is responsible for weakening the refractive power of the cornea and facilitating the laser to reach (be condensed at) an eye tissue such as the crystalline lens. The interface unit 220 of the embodiment is configured to cover at least part of the cornea without direct contact with the cornea. The interface unit 220 includes a cover glass (contact glass) 221 that serves as an optical member that covers the cornea, and a support part 222 that supports the cover glass 221. The interface unit 220 is placed in the second holding unit (second holding part) 260 through a proximal part 223 formed at a proximal end of the support part 222.

The cover glass (cover) 221 is a member that covers the cornea. The cover glass 221 has at least a size sufficient to cover NA on which a laser spot is condensed. The cover glass 221 is a transparent member capable of light transmission. The material of the cover glass 221 contains, for example, glass or resin. In the embodiment, the shape of the cover glass 221 is a planar shape (plate shape). The cover glass 221 is located at a liquid surface of a liquid, which is described below, and is responsible for covering the liquid. The support part 222 is formed into a conical shape and is a member having a tapered part. The support part 222 supports the cover glass 221 in an area corresponding to the distal end of the cone. A fitting part is formed at the proximal part 223. The fitting part enables the proximal part 223 and the second holding unit 260 to be fitted to each other. In other words, the proximal part 223 is detachably held by the second holding unit 260.

The interface unit 220 has a shape to be accommodated inside the suction ring 210. The materials of the members of the interface unit 220 contain a biocompatible material. The interface unit 220 is a disposable type similarly to the suction ring 210.

The second holding unit (interface unit holding unit) 260 is provided to the main body of the apparatus similarly to the first holding unit 250. The second holding unit 260 is arranged in the vicinity of the objective lens 150. The second holding unit 260 is arranged inside the first holding unit 250. The second holding unit 260 includes a holding part (fitting part) 261, a screw (shaft) 263, and a driving part 264.

The holding part 261 supports the support part 222 by fitting the proximal part 223 of the support part 222 thereto. The screw (guide mechanism) 263 guides the movement in the Z direction of a proximal part 262 formed at a proximal end of the holding part 261. The driving part 264 rotates the feed screw 263 to move the proximal part 262.

The holding part 261 is a ring-shaped member surrounding the objective lens 150. The holding part 261 holds the support part 222 detachably. Therefore, the holding part 261 is configured such that the holding part 261 and the support part 222 are fitted to each other. The proximal part 262 of the holding part 261 forms a flange of the holding part 261. The proximal part 262 of the holding part 261 is a ring-shaped member having a smaller diameter than that of the proximal part 252.

A female screw whose illustration is omitted is formed along the up-down direction on the proximal part 262. The female screw is threadedly engaged with the feed screw 263. The driving part 264 is, for example, a pulse motor. The feed screw 263 is fixed to a shaft of the driving part 264. Consequently, the feed screw 263 is axially rotated. The proximal part 262 is moved along the Z direction by the rotation of the feed screw 263. The proximal part 262 and the feed screw 263 constitute a moving mechanism. Moreover, the proximal part 262, the feed screw 263, and the driving part 264 constitute a moving unit (alignment unit) that enables the interface unit 220 and the second holding unit 260 to move.

The driving part 264 is connected to the control unit 70. The driving part 264 moves the holding part 261 based on an instruction signal of the control unit 70. Consequently, the interface unit 220 moves along the Z direction. The control unit 70 transmits to the driving part 264 a signal corresponding to an operation input into the operating unit 400 (an operation signal) as an instruction signal. Consequently, the control unit 70 moves the interface unit 220. In the embodiment, the operator uses an operating unit 400 to determine the position of the cover glass 221.

The interface unit 220 comes into contact with the cornea of the patient's eye E suctioned to the suction ring 210. Then, a liquid (normal saline) LQ is filled in the suction ring 210. The cover glass 221 comes into contact with the cornea through the liquid without a direct contact with the cornea. The refractive power of the cornea is cancelled by the cover glass 221 and the liquid LQ. Therefore, the laser is guided from the objective lens 150 to the crystalline lens being a target without refraction.

The interface unit 220 may be configured to come into direct contact with the cornea. In the interface unit 220, for example, the cover glass 221 may applanate the cornea by coming into contact with the cornea. Consequently, an eye tissue (corneal stroma) to be irradiated with the laser may be positioned. In this case, the cornea comes into direct contact with the cover glass 221 to determine the absolute position of the cornea with respect to the laser irradiation optical system. In this case, the cover glass 221 has a contact surface for covering the cornea to cover a laser irradiation area in the cornea or the like.

In this manner, the suction ring 210 and the interface unit 220 can move in the Z direction independently of each other. Moreover, in the embodiment, the suction ring 210 and the interface unit 220 do not move in the X and Y directions. Consequently, operations such as suction of the patient's eye E become easy.

The present apparatus 500 includes a (coarse) alignment unit (moving unit) 180. The alignment unit 180 adjusts the positional relationship between the laser irradiation unit 100 and the eye fixation/interface unit 200, and the patient's eye E (performs an alignment).

The alignment unit 180 has a moving mechanism for moving part of the main body such as the eye fixation/interface unit 200, and a driving part for driving the moving mechanism (a motor, an actuator, or the like). The alignment unit 180 moves (the optical axis L1 of) the laser irradiation unit 100 and (a central axis of) the eye fixation/interface unit 200 three-dimensionally with respect to the patient's eye E. In the embodiment, the optical axis L1 substantially coincides with the central axis of the eye fixation/interface unit 200. Moreover, a central axis of the suction ring 210 substantially coincides with the central axis of the interface unit 220.

The alignment unit 180 is connected to the control unit 70. The control unit 70 moves the laser irradiation unit 100 and the eye fixation/interface unit 200 by controlling the alignment unit 180 based on an operation signal from the operating unit 400. The operator performs alignments in the X and Y directions (XY alignments) and an alignment in the Z direction (a Z alignment) while checking the patient's eye E displayed on a monitor 420. In the embodiment, the laser irradiation unit 100 and the eye fixation/interface unit 200 move integrally.

The alignment unit 180 is an alignment unit that moves the suction ring 210 and the interface unit 220 along the Z direction. The alignment unit 180 includes a sensor such as an encoder therein. Consequently, the positions of the eye fixation/interface unit 200 and the like are obtained by the control unit 70. The alignment unit (alignment moving unit) 180 is configured to move the laser irradiation unit 100 and the eye fixation/interface unit 200 at least in the Z direction. For example, if the laser irradiation unit 100 and the eye fixation/interface unit 200 are attached to another apparatus (coupled to the laser irradiation unit 100), such as a surgical microscope, the XY alignment may be performed by the operator moving with the another apparatus.

<Observation and Photographing Unit>

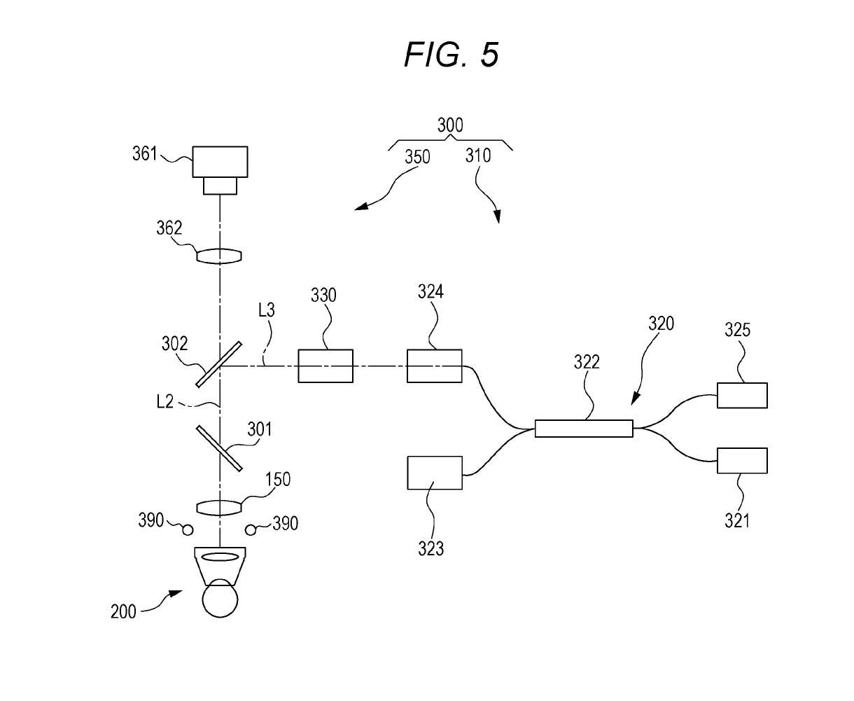

FIG. 5 is a block diagram of the observation and photographing unit 300. As illustrated in FIGS. 1 and 5, the observation and photographing unit 300 includes the OCT unit 310 that obtains a tomographic image of the patient's eye E, and the front observing unit (front image acquisition unit) 350 that obtains a front image of the patient's eye E. An optical axis L2 of the observation and photographing unit 300 is made substantially coaxial (or substantially parallel) with the laser optical axis L1 by the beam combiner 301. It is set as the optical axis L2 of the observation and photographing unit 300. The optical axis L2 is divided by a beam combiner 302 to cause an optical axis L3 of the OCT unit 310. Here, the beam combiner 302 is a dichroic mirror. The beam combiner 302 has a characteristic to transmit measurement light of the OCT unit 310 while transmitting (the reflected light of) illuminating light for the front observing unit 350.

<Optical Tomography Unit>

The optical coherence tomography unit (OCT unit) 310 and the laser irradiation unit 100 (the laser irradiation optical system 120) share the objective lens 150. The OCT unit 310 includes a coherent optical system (OCT optical system) 320 for capturing a tomographic image of (the anterior segment of) the patient's eye E.

The OCT optical system 320 irradiates the patient's eye E with measurement light. The OCT optical system 320 detects a coherent state of the measurement light reflected from the patient's eye E and reference light, by a light receiving device (a detector 325). The OCT optical system 320 includes an optical scanner 330 to change the imaging position of the patient's eye E. The optical scanner 330 is an irradiation position changing unit that changes the irradiation position of the measurement light at the fundus (patient's eye) E. The optical scanner 330 is connected to the control unit 70. The control unit 70 controls the operation of the optical scanner 330 based on the set imaging position information. Consequently, the control unit 70 obtains a tomographic image based on a light receiving signal from the detector 325.

The OCT optical system 320 has an apparatus configuration of what is called an ophthalmic optical coherence tomography. In the embodiment, the OCT optical system 320 captures at least a tomographic image of the patient's eye E before pulse laser is irradiated. The OCT optical system 320 divides light (infrared-light) emitted from a measurement light source 321 by a coupler (light splitter) 322 into measurement light (sample light) and reference light. The OCT optical system 320 then guides the measurement light to the patient's eye E by a measuring optical system 324 while guiding the reference light to a reference light optical system 323. The OCT optical system 320 receives, by the detector (light receiving device) 325, interference light obtained by combining the measurement light reflected by the patient's eye E and the reference light.

The detector 325 detects a coherent state of the measurement light and the reference light. In Fourier domain OCT, spectral intensity of the interference light is detected by a detector. A depth profile (A-scan signal) in a predetermined area is obtained by the Fourier transform on spectral intensity data. Fourier domain OCT includes, for example, Spectral-Domain OCT (SD-OCT) and Swept-Source OCT (SS-OCT). Moreover, Fourier domain OCT may be Time-Domain OCT (TD-OCT).

Light emitted from the light source 321 is divided by the coupler into a measurement light flux and a reference light flux. The measurement light flux is emitted into the air after passing through an optical fiber. The light flux is condensed at the patient's eye through the measuring optical system 324 and the optical scanner 330. The light reflected by the patient's eye E is returned to the optical fiber through a similar optical path. The reference optical system 323 generates reference light. The reference light is combined with reflected light obtained by the reflection of the measurement light at the patient's eye E.

The reference optical system 323 includes a member for changing a difference in light wavelength between the measurement light and the reference light by moving an optical member on a reference optical path. For example, a reference mirror is moved in the optical axis direction to change the difference in light wavelength. The member for changing the difference in light wavelength may be arranged on a measurement optical path of the measuring optical system 324.

The OCT unit 310 includes the optical scanner 330 for deflecting the measurement light flux. The optical scanner 330 includes two galvanometer mirrors whose rotation axes are orthogonal to each other. The optical scanner 330 has a function of deflecting the measurement light flux two-dimensionally based on an instruction signal from the control unit 70. The optical scanner 330 scans the patient's eye E with the measurement light in the X-Y direction (transverse direction). In the embodiment, the anterior segment of the patient's eye E is scanned with the measurement light. For example, the control unit 70 may operate the optical scanner 330 linearly (for example, in the Y direction). The control unit 70 then linearly arranges depth information (depth information) obtained by the detector 325. Consequently, the control unit 70 obtains a tomographic image (what is called B-scan).

In this manner, a light flux emitted from a light source is changed in terms of its reflection (advance) direction and scans the anterior segment in an arbitrary direction. Consequently, the imaging position of the patient's eye E is changed.

The optical scanner 330 is configured to deflect light. The optical scanner 330 may include, for example, a reflection minor (a galvanometer mirror, a polygon minor, a resonant scanner) or may include an acousto-optic modulator (AOM) that changes (deflects) the advance direction of light, and the like. See, for example, JP-A-2008-29467 for the detailed configuration of the OCT unit 310.

<Front Observing Unit>

The front observing unit 350 has a function of acquiring a front image of the anterior segment of the patient's eye E. In the embodiment, the front observing unit 350 photographs an anterior segment image of the patient's eye E illuminated with visible light and displays the image on a monitor, which is described below. The front observing unit 350 includes an observing optical system (a front image observing optical system). The observing optical system includes a camera unit 361 having a two-dimensional imaging device, and a relay lens 362 for relaying an observation image. The front observing unit 350 and the laser irradiation unit 100 (the laser irradiation optical system 120) share the objective lens 150. Moreover, an illuminating light source 390 that emits visible illuminating light is arranged in the vicinity of the front of the patient's eye E. The captured front image is sent to the control unit 70.

<Operating Unit>

The operating unit 400 includes a trigger switch 410 and a monitor 420. A trigger signal to cause the laser irradiation unit 100 to emit treatment laser is input into the trigger switch 410. The monitor (display unit) 420 displays a tomographic image and/or anterior segment image of the patient's eye E, and surgical conditions. The monitor 420 has a touchscreen function. The monitor 420 serves also as an input unit (input part) for carrying out the setting of the surgical condition and/or the setting of a surgical site on a tomographic image. The input unit may be a mouse being a pointing device, and/or a keyboard being an input device for inputting numerical values, characters, and the like.

The monitor 420 includes an anterior segment display part 430 for displaying the anterior segment of the patient's eye E, an OCT image display part 440 for displaying a tomographic image of the anterior segment of the patient's eye E, a surgical condition display part 450 for displaying the surgical conditions, an eye fixation operating part (eye fixation/interface operating part) 460 for performing an operation for eye fixation, and a moving unit operating part 470 for operating the movement of the laser irradiation unit 100 and the like.

On the OCT image display part 440, the operator specifies a surgical site (a laser irradiation area) graphically. A signal corresponding to the surgical site specified on the monitor 420 is transmitted to the control unit 70 as a signal to specify an area on the OCT image.

On the surgical condition display part 450, the operator sets an irradiation pattern of treatment laser to fragment (incise) the crystalline lens. There is a plurality of irradiation patterns prepared in advance. The operator selects one of the irradiation patterns. When the irradiation pattern is set by the surgical condition display part 450, the monitor 420 transmits a setting signal to the control unit 70. Laser output, the spot size of a laser spot, and the like may not be changed or may be changed (set) by the operator.

The OCT image display part 440 (the monitor 420) graphically displays the suction ring 210 and the interface unit 220 on the OCT image. Consequently, it becomes easy for the operator to visually perform alignment of the suction ring 210 and the interface unit 220.

In the embodiment, the OCT image is displayed as a moving image on the OCT image display part 440 at least until the positioning of the suction ring 210 and the interface unit 220 is complete. In the embodiment, the cornea of the patient's eye E and the vicinity of the cornea (the sclera in the vicinity of the ring), and the suction ring 210 (the ring 211) are captured and shown in the OCT image. Upon the positioning of the suction ring 210 and the interface unit 220, an OCT image at the time of eye fixation includes at least the cornea of the patient's eye E and the ring 211. Upon the positioning of the cover glass 221, the OCT image includes at least the cornea and the cover glass 221.

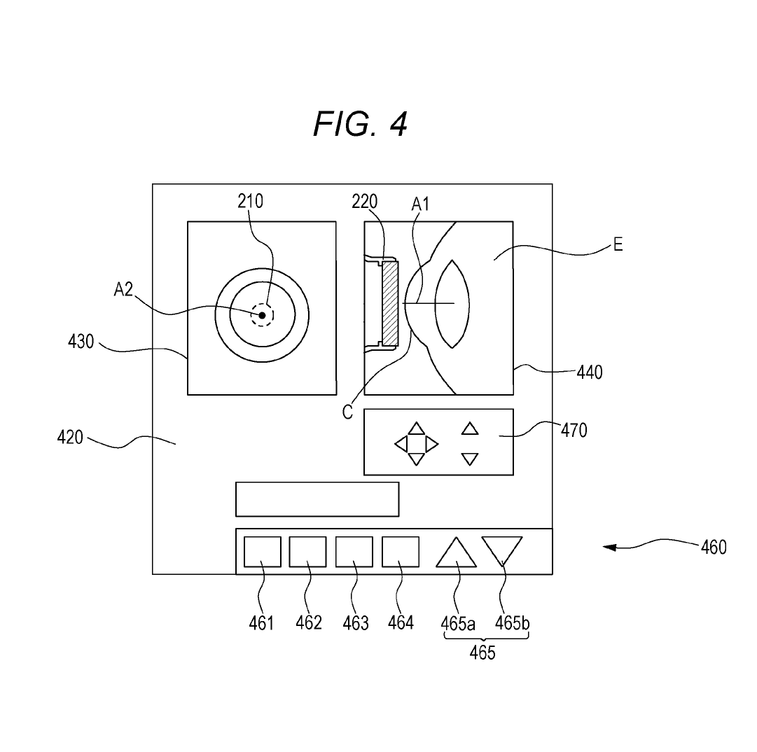

As illustrated in FIG. 4, the eye fixation operating part 460 includes a suction pressure setting part 461, a suction switch 462, a supply switch 463, a drain switch 464, and a vertical motion switch 465.

The suction pressure setting part 461 sets a suction pressure to be added to the suction ring 210 (the ring 211). The suction switch 462 is a switch for inputting an instruction signal to suction the eye by the suction ring 210. The supply switch 463 is a switch for inputting an instruction signal to supply liquid into the suction ring 210. The drain switch 464 is a switch for inputting an instruction signal to drain the liquid from inside the suction ring 210. The vertical motion switch 465 is a switch for adjusting the position (height position) of the interface unit 220.

When the suction pressure setting part 461 is operated, a numeric keypad for setting the numeric value of a suction pressure is displayed. The numeric value input by the operator is stored as a set value in a memory 71 (described below). The control unit 70 controls the suction pump based on the set suction pressure.

When the suction switch 462 is operated, the suction pressure to be added (applied) to the ring 211 is turned on/off. When the supply switch 463 is operated, an instruction signal is transmitted to the control unit 70. The control unit 70, which has received the instruction signal, controls the irrigation suction unit and supplies liquid into the suction ring 210 through the pipe 215 such that the water level of the liquid in the suction ring 210 is at a predetermined water level.

When the drain switch 464 is operated, an instruction signal is transmitted to the control unit 70. The control unit 70, which has received the instruction signal, controls a suction/drain unit and drains the liquid in the suction ring 210 through the pipe 215. The vertical motion switch 465 includes an upward cursor 465a and a downward cursor 465b. When the cursor 465a is operated, an instruction signal (operation signal) for moving the interface unit 220 upward along the Z direction is transmitted to the control unit 70. The control unit 70, which has received the signal, controls the driving part 264 and moves the cover glass 221 upward. On the other hand, when the cursor 465b is operated, the control unit 70 moves the cover glass 221 downward.

Although details are described below, the operator moves the fixation unit while watching the moving image of the patient's eye E, the moving image being displayed on the monitor 420, and fixes the eye. Therefore, the monitor (part of a monitoring unit) 420 is included in a member for adjusting the position of the fixation unit.

The moving unit operating part 470 is an input unit for inputting into the alignment unit 180 an instruction signal (operation signal) to move in the X, Y, and Z directions. The operating unit 270 is provided with cursors arranged in the plus and minus directions for each of the X, Y, and Z directions. When the cursor is operated, an instruction signal regarding a direction corresponding to the operated cursor is transmitted to the control unit 70.

<Control System>

The control unit 70 oversees and excises control (including display control and drive control) over the entire present apparatus 500. The control unit 70 is, for example, a CPU (Central Processing Unit). The control unit 70 is connected to the laser light source unit 110, the expander 130, the scanning unit 140, the position detecting unit 190, the regulation part 255, the sensor 256, the driving part 264, the OCT unit 310, the front observing unit 350, the operating unit 400 (the trigger switch 410 and the monitor 420), the alignment unit 180, the suction pump, and the irrigation suction unit.

Moreover, the control unit 70 is connected to the memory 71. The memory 71 stores therein the surgical conditions, the irradiation patterns (patterns to move a laser spot), a control program of the eye fixation/interface unit 200, and the like. Moreover, the control unit 70 is connected to a buzzer 72. The buzzer 72 notifies the operator of the end of an operation and/or alert. The eye fixation/interface unit 200 and the illuminating light source 390 are driven individually.

After the fixation of the patient's eye E by the eye fixation/interface unit 200 is complete, the control unit 70 uses the position detecting unit 190 and obtains the absolute position of the feature portion (anterior lens capsule) of the patient's eye E. The control unit 70 corrects (aligns) the laser irradiation position based on the acquisition result.

Before the irradiation of the surgical laser, the control unit 70 corrects the position information for the irradiation of the surgical laser, based on the surgical site (region) set using the OCT image display part 440, and the absolute information obtained using the position detecting unit 190. The control unit 70 emits laser from the laser light source unit 110 based on the corrected surgical site, the surgical condition, and the irradiation pattern. Furthermore, the control unit 70 controls the expander 130 (a driving part 135) and the scanning unit (galvanometer mirrors 141 and 144), moves a laser spot on an eye tissue, and incises and breaks up a predetermined eye tissue.

The control unit 70 controls the alignment unit 180 based on an instruction signal from the moving unit operating part (eye fixation operating part) 470. Consequently, the control unit 70 moves the eye fixation/interface unit 200, together with the laser irradiation unit 100, in the X, Y, and Z directions. Consequently, the control unit 70 performs alignments in the X and Y directions of and a coarse alignment in the Z direction of the eye fixation/interface unit 200.

The control unit (display control unit) 70 performs image processing on a tomographic image obtained by the OCT unit 310 and the like. Based on the image processing result, the control unit 70 performs control of the eye fixation/interface unit 200, acquisition (detection and calculation) of the position and shape of a tissue of the patient's eye E, and display (display control). Moreover, the control unit 70 displays a tomographic image on the OCT image display part 440 based on the image processing result, or the control unit 70 controls the eye fixation/interface unit 200 based on the image processing result. Consequently, the control unit 70 monitors the position of the eye fixation/interface unit 200. In other words, the control unit 70 serves as (at least part of) a function of the monitoring unit.

<Flow of Surgery>

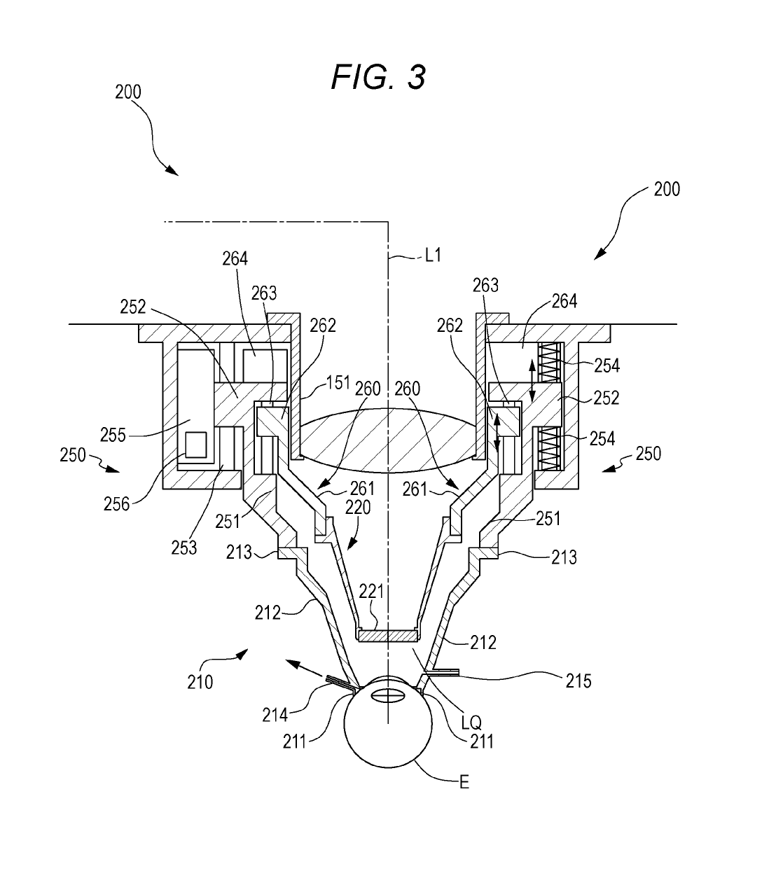

Next, the flow of surgery will be described focusing on an eye fixation operation. FIG. 3 illustrates a state where the interface unit 220 of the eye fixation/interface unit 200 is at an uppermost part. FIG. 4 illustrates the OCT image display part 440.

The operator attaches the suction ring 210 to the holding part 251, and the interface unit 220 to the holding part 261. The operator sets the surgical condition by operating the surgical condition display part 450 of the monitor 420. The operator selects, for example, the irradiation pattern for fragmenting the crystalline lens. The irradiation patterns include, for example, a pattern for incising only the anterior capsule of the crystalline lens, a pattern for incising the anterior capsule and segmenting the crystalline lens nucleus (for example, into two, four, or eight), and a pattern for incising the anterior capsule and fragmenting the crystalline lens nucleus into pieces. Moreover, the operator uses the suction pressure setting part 461 and sets a suction pressure at the time of eye fixation. A signal for setting the irradiation pattern and a signal for setting the suction pressure are transmitted to the control unit 70 and stored in the memory 71.

Next, the operator aligns the patient's eye E of a patient (person to be operated) who is lying on a bed or the like with the optical axis L1 of the main body of the apparatus (XY alignment). The operator subsequently moves the main body of the apparatus in the Z direction. Consequently, the suction ring 210 (the ring 211) is brought close to the sclera of the patient's eye E. Then, the operator works while watching moving images displayed on the anterior segment display part 430 and the OCT image display part 440. The operator checks the contact state of the suction wing 210 with the sclera while watching the moving images (a front image and an OCT image). The control unit 70 performs image processing on the OCT image, the moving image obtained by the OCT unit 310. Consequently, the control unit 70 extracts the corneal shape of the patient's eye E and extracts the position of the corneal apex.

Moreover, the control unit 70 extracts the iris of the patient's eye E to obtain the position of the pupil center. The control unit 70 then recognizes a line passing the corneal apex and the pupil center as a direction of the patient's eye E (axial information indicating the axis of the eye). The control unit 70 superimposes and displays an axis indication symbol (mark) A1 on the OCT image. Moreover, the control unit 70 performs image processing on the front image obtained by the front observing unit 350. Consequently, the control unit 70 extracts the position of the pupil center. The control unit 70 superimposes and displays a pupil-center position indication symbol A2 on the front image. The symbols A1 and A2 are displayed while updated in real time. Consequently, the operator can check the direction of the patient's eye E by watching the monitor 420. Therefore, the operator can easily align the central axis of the patient's eye E with the center of the suction ring 210. The symbol A2 may be a symbol indicating the position of the corneal apex. Moreover, the symbol A2 may be a symbol indicating the symbol A1 (the position of the corneal apex). In this case, it is preferred that the control unit 70 extract the position of the corneal apex for each of a plurality of OCT images corresponding to different B-scan directions.

Also after the suction ring 210 comes into contact with the sclera of the patient's eye E, the operator moves the main body of the apparatus in the Z direction. Then, the force absorbing mechanism of the first holding unit 250 makes it hard for the suction ring 210 to apply an excessive pressure to the patient's eye E. The control unit 70 monitors a detection signal of the sensor 256. Consequently, the control unit 70 determines whether or not the suction ring 210 is located within the alignment area. If determining that the suction ring 210 is located within the alignment area, the control unit 70 transmits an instruction signal to the regulation part 255. The regulation part 255 locks the movement of the suction ring 210 based on the instruction signal. The control unit 70 controls the buzzer 72 and notifies the operator that the movement of the suction ring 210 has been locked. The operator operates the suction switch 462 to suction the patient's eye E to the suction ring 210. The operator operates the supply switch 463 to fill liquid inside the suction ring 210. The control unit 70 may be configured to start suction at the same time as the locking of the movement of the suction ring 210. Moreover, the control unit 70 may be configured to supply liquid after the completion of suction of the suction ring 210.

Next, the operator adjusts the position of the interface unit 220 with respect to the cornea. In other words, the operator checks the position of the cover glass 221 (the height position in the Z direction) and the position of the cornea while watching the OCT image. At the same time, the operator operates the cursor 265b (or the cursor 265a) and accordingly adjusts the position of a front surface (lower surface) of the cover glass 221 so as to have a predetermined positional relationship between the position of the front surface of the cover glass 221 and the position of the cornea (apex). With regard to the positional relationship, the space between the corneal apex and the front surface of the cover glass 221 is set to approximately 1 mm. The space makes it possible to inhibit the cover glass 221 from receiving the influence of the shaking of the surface of the liquid and from coming into direct contact with the cornea and placing a burden on a patient.

The control unit 70 transmits to the driving part 264 an operation signal input by the operating unit 400 as an instruction signal. The control unit 70 moves the interface unit 220 in the Z direction with respect to the examinee's eye by driving the driving part 264. When the operation signal from the operating unit 400 is turned off, the control unit 70 stops the drive of the driving part 264. Consequently, the movement of the interface unit 220 stops. The control unit 70 may be configured to detect the position of the front surface of the cover glass 221 and the position of the corneal apex by the image processing of an OCT image and to set the position of the cover glass 221 based on the detection result.

In this manner, the patient's eye E is fixed and the position of the cover glass 221 is adjusted. The operator can move the suction ring 210 and the interface unit 220 in the Z direction while watching moving images such as an OCT image. Consequently, the operator can easily adjust the position of the cover glass 221. Moreover, the operator can check the moving images of the patient's eye upon eye fixation. Consequently, the operator can execute eye fixation corresponding to the positions of the sclera and the cornea, which are different among patients' eyes. In other words, the operator can execute suitable eye fixation in accordance with the patient's eye.

Moreover, the present apparatus 500 includes the main body of the apparatus provided with the suction ring 210 and the interface unit 220. The present apparatus can move the suction ring 210 and the interface unit 220 independently in the Z direction. The present apparatus 500 moves the interface unit 220 with respect to a patient's eye fixed by the suction ring 210. Consequently, the relationship between the position of the patient's eye fixed by the suction ring 210 and the position of the position of the interface unit 220 can be suitably adjusted. Therefore, the operator can execute eye fixation corresponding to the positions of the sclera and the cornea, which are different among patients' eyes. In other words, the operator can execute suitable eye fixation dependent on the patient's eye. Furthermore, the movement of the interface unit 220 in the X and Y directions is regulated by the guide mechanism in the Z direction. The operator or the control unit 70 can move such an interface unit 220 in the Z direction with respect to the suction ring 210. Therefore, the interface unit 220 and the suction ring 210 can be prevented from deviating in the X and Y directions.

Moreover, the present apparatus 500 includes the main body of the apparatus provided with the suction ring 210 and the interface unit 220. The present apparatus can move the suction ring 210 and the interface unit 220 independently in the Z direction. Thus, the present apparatus 500 can smoothly fix the suction ring 210 to a patient's eye and adjust the positional relationship between the patient's eye fixed by the suction ring 210 and the interface unit 220. Consequently, the operator can carry out surgery efficiently.

When the eye fixation is complete, the operator carries out the setting (planning) of a surgical site. The operator operates the switch of the OCT image display part 440 and carries out planning. When the planning is complete, the control unit 70 controls the position detecting unit 190 and accordingly obtains the absolute position of a feature portion of eye tissue of the patient's eye E. The control unit 70 corrects laser irradiation position information based on the obtained absolute position. The correction result is stored in the memory 71.

When the operator operates the trigger switch 410, the control unit 70 performs laser irradiation (control of the laser irradiation optical system 120) dependent on the set surgical condition and irradiation pattern, and the corrected laser irradiation position information in response to the trigger signal.

The crystalline lens of the patient's eye is incised and broken up by the laser irradiation. Furthermore, the anterior lens capsule is incised. When the laser irradiation is complete, the control unit 70 notifies the operator of the completion of the laser irradiation by the buzzer 72. The operator operates the cursor 465a to move the interface unit 220 upward. Moreover, the operator operates the drain switch 464 to drain the liquid in the suction ring 210. The operator then operates the suction switch 462 to release the suction by the suction ring 210. Consequently, the suction ring 210 is removed from the patient's eye E. The control unit 70 may be configured to perform processing after the completion of the laser irradiation (upward movement of the interface unit 220, drain of the liquid, and release of the suction by the suction ring 210).

After the eye fixation/interface unit 200 is removed, the patient's eye is operated by another surgical apparatus, for example, an ultrasonic cataract surgical apparatus.

In the above description, the suction ring 210 and the interface unit 220 are a disposable type (one use type). However, it is sufficient if they can fix an eye, and they may be a reusable type. In this case, the suction ring 210 and the interface unit 220 are formed of materials that can be repeatedly sterilized (for example, stainless and steel).

In the embodiment described above, upon eye fixation, the interface unit 220 is brought into contact with a patient's eye after the suction of the patient's eye by the suction ring 210. However, upon eye fixation, the suction ring 210 may suction a patient's eye after the interface unit 220 is brought into contact with the patient's eye.

In the above description, the suction ring 210 and the interface unit 220 may be configured to move in the Z direction. However, the movement direction of the suction ring 210 and the interface unit 220 is not limited to the Z direction. It is sufficient if the suction ring 210 and the interface unit 220 are configured to move independently. The suction ring 210 and the interface unit 220 may be configured to move (or rotate) in the X and Y directions.

In the above description, the suction ring 210 is configured to move, but not limited thereto. Alternatively, the suction ring 210 and the interface unit 220 may be configured to move relative to each other. For example, the mechanism for moving the suction ring 210 in the Z direction is not necessarily required. Similarly, a mechanism for moving the interface unit 220 in the Z direction is not necessarily required. The interface unit 220 may be fixed to the laser irradiation optical system 120.

In the above description, the position of the suction ring (fixation unit) 210 is adjusted using an image (a moving image) captured as an OCT image, but not limited thereto. Alternatively, for example, at least a symbol (an illustration, a frame or the like) indicating the position of the ring 211 of the suction ring 210 may be superimposed and displayed on the OCT image. For example, the control unit 70 may be configured to obtain the position (position information) of the ring 211 of the suction ring 210 from the position (configuration) of the member of the suction ring 210 and/or a detection signal of the sensor 256 of the first holding unit 250 and display the position of the ring 211 on an OCT image. Consequently, the operator can check on the monitor 420 the position of a member that is hard to be shown in an image.

It is preferred that the control unit 70 indicate at least the position of a contact area of the suction ring 210 with a patient's eye or the position of an area on the patient's eye side of the interface unit 220 (the cover glass 221) with a symbol indicating the position of the suction ring 210 or the interface unit 220.

Moreover, the control unit (monitoring unit) 70 may be configured to detect the position information of the suction ring (fixation unit) 210 and/or the interface unit 220 based on a tomographic image. In this case, the control unit 70 may display on the monitor 420 a symbol(s) indicating the suction ring (fixation unit) 210 and/or the interface unit 220 based on the detected position information of the suction ring (the fixation unit) 210 and/or the interface unit 220.

Moreover, the control unit (the eye shape acquisition unit and the eye position acquisition unit) 70 may be configured to extract the shape of a patient's eye from an OCT image by image processing and the like and use the extraction result for eye fixation and the like. For example, the control unit 70 obtains an OCT image (or an OCT image obtained by another photographing apparatus) before the eye fixation operation. The control unit 70 performs image processing on the OCT image, and previously extracts the corneal shape of the patient's eye and the shape (shape of the eye) of the vicinity of the cornea that comes into contact with the suction ring 210. It is preferred that the shape of the eye be a three-dimensional shape obtained by a scan (B-scan) regarding an orthogonal line passing the vicinity of the corneal apex.

For example, the control unit 70 obtains the corneal shape of a patient's eye upon the eye fixation operation. Furthermore, the control unit 70 performs matching between the corneal shape obtained upon the eye fixation operation and the previously-obtained corneal shape. Consequently, the control unit 70 obtains the shape and position of the vicinity of the cornea of the patient's eye. The control unit 70 may be configured to display the obtained shape and position of the vicinity of the cornea as a symbol on an OCT image. Consequently, even if the vicinity of the cornea cannot be captured by the OCT unit 310, the operator can adjust the position of the suction ring 210 while checking (estimating) the position of the vicinity of the cornea. In the acquisition of an OCT image of a patient's eye, the apparatus (the OCT unit 310) may be moved in the X and Y directions, for example, before the eye fixation operation. The control unit 70 may display a symbol indicating the patient's eye on the monitor 420 based on the obtained shape of the eye. Moreover, the control unit 70 may be configured to acquire the position information of the patient's eye based on the obtained shape of the eye and the tomographic image captured by the tomography unit.

In the above description, the OCT unit 310 is configured to acquire an OCT image including at least part of the suction ring (fixation unit) 210 or the interface unit 220, but not limited thereto. Alternatively, it is sufficient if an OCT image is an image where the positions and shapes of the fixation unit and the like can be recognized. For example, illustrations or the like that indicate parts of the suction ring (fixation unit) 210 and the like may be displayed as symbols on an OCT image. Specifically, the control unit 70 obtains the position of the suction ring 210 based on information (position detection results) from the sensor 256 and the alignment unit. The control unit 70 displays an illustration mimicking the suction ring 210 on an OCT image. The illustration is obtained from the design information of the suction ring 210. The control unit 70 updates the display based on the position information of the sensor. The operator can know the position of the suction ring 210 by the illustration on the OCT image, which facilitates alignment. Also in the case of the interface unit 220, information from the driving part 264 and the alignment unit is similarly obtained. The control unit 70 displays an illustration of the interface unit 220 (the illustration at least indicating the cover glass 221) on the OCT image. Also if parts of the suction ring (fixation unit) 210 and the interface unit 220 are included in the OCT image, the control unit 70 may perform the above symbol display.

The tomography unit (the OCT unit 310) may be configured to include at least part of the suction ring (fixation unit) 210 and/or the interface unit 220 in a tomographic image to be captured. Moreover, the control unit (monitoring unit) 70 may display on the monitor 420 at least part of the suction ring (fixation unit) 210 and/or the interface unit 220, the part having been captured by the tomography unit, to graphically indicate the position of the patient's eye and the position of the suction ring (fixation unit) 210 and/or the interface unit 220.

In the above description, alignment of the suction ring 210 and the interface unit 220 is performed by the operation of the operator, but not limited thereto. Alternatively, it is sufficient if the present apparatus 500 is configured to be capable of performing alignment of each unit. For example, the control unit 70 may be configured to identify the position of the patient's eye based on an OCT image and the like, and adjust the positions of the suction ring (fixation unit) 210 and the like. Specifically, the control unit 70 obtains the position of the suction ring 210 based on the design information of the suction ring (suction ring unit) 210 and the detection result of the sensor 256 in the suction ring 210. Moreover, the control unit 70 obtains the position of the ring that comes into contact with the suction ring 210 from the OCT image. Otherwise, the control unit 70 estimates the position of the ring from the corneal shape. Moreover, the control unit 70 may be configured to previously obtain information on the axis of the patient's eye, and use information including the axial information so as to perform alignment of the suction ring 210 in the X, Y, and Z directions and the suction operation. The case of the interface unit 220 is also similar. In other words, the control unit 70 may be configured to perform alignment of the interface unit 220 in the Z direction based on the position of the cornea (apex) of the patient's eye and the shape and position of the cover glass 221, and the preset positional relationship between the cornea and the cover glass 221 (the height of the cover glass 221).

Moreover, the control unit 70 may be configured to adjust the XY position of the eye fixation/interface unit (the laser irradiation unit 100) 200. The control unit 70 calculates a difference in the X-Y direction between the corneal apex or pupil center position obtained from an OCT image and the central axis (the optical axis L1) of the eye fixation/interface unit 200. The control unit 70 controls the alignment unit and moves the eye fixation/interface unit 200 so as to eliminate the difference of the calculation result. Then, the control unit 70 controls the alignment unit such that the axis of the patient's eye coincides with the central axis. The control unit 70 may be configured to guide the axis (visual line) of the patient's eye relatively or move the patient's eye for alignment for eye fixation and the like.

For example, the control unit 70 may guide the visual line of the patient's eye by controlling a fixation light arranged to coincide with the optical axis of the laser irradiation optical system 120. Moreover, the control unit 70 may move a bed where the patient is lying or a headrest (pillow) in an inclined and/or parallel direction(s) and the like. Moreover, the control unit 70 may promote the movement of the visual line of the patient's eye or of the patient by audio guidance.

In the present apparatus 500, the control unit (monitoring unit) 70 may acquire the position of the corneal apex of a patient's eye or the position of the pupil center of the patient's eye from a tomographic image captured by the tomography unit (the OCT unit 310). Furthermore, the control unit 70 may display a symbol indicating the obtained position on the tomographic image. Moreover, the control unit 70 may display the symbol indicating the obtained position on a front image captured by the front observing unit (front image photographing unit) 350.

Furthermore, the control unit 70 may obtain displacement information indicating a deviation between the obtained position of the corneal apex or the pupil center and the central axis of the suction ring (fixation unit) 210 or the interface unit 220. In this case, it is preferred that the moving unit (alignment unit) be capable of moving the suction ring (fixation unit) 210 or the interface unit 220 along a two-dimensional plane substantially orthogonal to a depth direction of the patient's eye. Furthermore, the control unit 70 may control the moving unit based on the obtained displacement information and cause the position of the corneal apex or the pupil center to coincide (substantially coincide) with the central axis of the suction ring (fixation unit) 210 or the interface unit 220.

Moreover, in the present apparatus 500, the control unit 70 (the monitoring unit) 70 may acquire axial information on an axis passing the corneal apex of a patient's eye or an axis passing the pupil center of the patient's eye based on a tomographic image captured by the tomography unit (the OCT unit 310). Furthermore, the control unit 70 may acquire the direction of the patient's eye based on the obtained axial information. The control unit 70 may obtain displacement information between the patient's eye and the central axis of the fixation unit or the interface unit based on the axial information and the direction of the patient's eye.

In the above description, alignment is performed such that the axis of the patient's eye coincides with the optical axis of the laser irradiation optical system 120, but not limited thereto. Alternatively, the present apparatus 500 (the control unit 70) may be configured that an alignment reference can be changed (an offset can be added) dependent on the state of the patient's eye and/or the technique.

For example, the control unit 70 may be configured to perform alignment such that the optical axis of the laser irradiation optical system 120 coincides with between the corneal apex and pupil center of the patient's eye. Moreover, the control unit 70 may be configured to perform alignment such that the patient's eye is inclined.

Such offset information is decided by a preoperative examination and the like, and/or a technique. Upon performing an alignment corresponding to an offset, a display unit such as a liquid crystal monitor is used. The monitor 420 is arranged to be coaxial with the laser irradiation optical system 120. A mark serving as a fixation target and the like are displayed on the monitor 420. A mark is displayed at the position corresponding to an offset of the patient's eye, the position being away from the optical axis of the laser irradiation optical system 120. Consequently, the visual line of the patient's eye can be guided. The visual line of the patient's eye and/or the position of the patient's eye can be similarly changed by a bed, a headrest, audio guidance, or the like.

The tomography unit (the OCT unit 310) may be configured to show at least part of the interface unit 220 in a tomographic image to be captured. In this case, the control unit (monitoring unit) 70 may be configured to extract the position of the interface unit 220 and the position of the patient's eye from a tomographic image captured by the tomography unit and control the moving unit based on the extraction result.

Moreover, the monitoring unit in the present apparatus 500 may include the sensor 256 for detecting the position information of the suction ring (fixation unit) 210 or the interface unit 220. In this case, the control unit (eye position acquisition unit) 70 obtains the position information of the patient's eye based on a tomographic image captured by the tomography unit (the OCT unit 310). Furthermore, the control unit 70 controls the moving unit based on the position information of the suction ring (fixation unit) 210 or the interface unit 220, the position information having been detected by the sensor 256, and the position information of the patient's eye.

In the above description, a moving image of an OCT image is displayed on the monitor 420, but not limited thereto. Alternatively, it is sufficient if an OCT image is an image that the control unit 70 can achieve the monitoring when alignment of the eye unit and the like is performed. The captured OCT image may be processed inside. In this case, the control unit 70 functions as the monitoring unit.

In the above description, the OCT unit 310 is used as the tomography unit, but not limited thereto. Alternatively, it is sufficient if the tomography unit can photograph a tomographic image that permits the acquisition of depth information of the patient's eye. For example, the tomography unit may be a Scheimpflug camera unit.

In the above description, an OCT image and the like are used for alignment of the suction ring (fixation unit) 210 before laser irradiation (before surgery), but not limited thereto. Alternatively, the control unit 70 may monitor (check) the state of eye fixation using an OCT image during laser irradiation (during surgery). For example, the control unit 70 detects the movement of the patient's eye from an OCT image by image processing. The control unit 70 previously obtains information on a feature portion of the patient's eye, such as the corneal shape or the position of the corneal apex, from the OCT image. The control unit (eye movement detecting unit) 70 monitors the position of the feature portion and detects the movement of the feature portion. For example, if the eye ends up moving by a suction break or the like, the control unit 70 is configured to stop the laser irradiation based on the detection result and notify the operator. In such a case, a suction state can be detected faster than a case of detecting a suction state using a sensor for monitoring the suction pressure of the suction ring 210. Moreover, a detection target is not limited to a suction state. The control unit 70 may detect a change of the patient's eye from an OCT image and the like. The control unit 70 may be configured to stop the laser irradiation or correct (the position of) the laser irradiation, based on the detection result.

In the above description, at least part of the suction ring (fixation unit) 210 and/or at least part of the interface unit 220 is/are shown in an OCT image, but not limited thereto. Alternatively, it is sufficient if an OCT image is an image that permits alignment of the interface unit 220. It is sufficient if an OCT image includes part of the interface unit 220. Consequently, it is easy to acquire the positional relationship between the cornea and the interface unit 220 in image display and image processing.

In the above description, at least part of the suction ring (fixation unit) 210 and/or at least part of the interface unit 220 is/are shown in an OCT image, but not limited thereto. Alternatively, it is sufficient if an OCT image is an image that permits alignment of the fixation unit and the interface unit 220 with respect to the cornea. The units are not necessarily shown in an OCT image. The positional relationship between the OCT unit 310 and the cornea (surface) can be obtained from a captured OCT image. Hence, determination of the positional relationship between the OCT unit 310, the suction ring (fixation unit) 210, and the interface unit 220 enables acquisition of the positional relationship between the cornea (patient's eye) and the suction ring (fixation unit) 210 and alignment of the units, by using an OCT image where the suction ring (fixation unit) 210 and the like are not shown.

In the above description, in alignment of the suction ring (fixation unit) 210 and the interface unit 220, the alignment using an OCT image, the units are arranged fixedly to the main body of the apparatus, but not limited thereto. Alternatively, it is sufficient if the present apparatus 500 is configured to fix an eye to the laser irradiation unit 100. For example, the suction ring (fixation unit) 210 may be a unit independent of the present apparatus 500. In this case, it facilitates positioning of the suction ring (fixation unit) 210 in the eye fixation work by using an OCT image and the like.

In the above description, the fixation unit (the suction ring 210) sucks an eye to suction and fix the eye thereon. However, the suction ring (fixation unit) 210 is not limited to this, but it is sufficient if the suction ring (fixation unit) 210 is configured to be capable of fixing an eye thereto. For example, the suction ring (fixation unit) 210 may be configured to come into contact with an eye and inhibit the movement of the eye.

In the above description, the ophthalmic laser surgical apparatus (the present apparatus 500) including pulse laser is illustrated as an example of an ophthalmic laser surgical apparatus. However, the ophthalmic laser surgical apparatus according to the embodiment is not limited to this. It is sufficient if the ophthalmic laser surgical apparatus according to the embodiment is configured to perform surgery or treatment by fixing a patient's eye (an eye to be operated) and irradiating an eye tissue of the fixed patient's eye with laser. The ophthalmic laser surgical apparatus according to the embodiment may be an ophthalmic laser surgical apparatus, for example, for performing selective laser trabeculoplasty (Selective Laser Trabeculoplasity). In this case, laser is visible pulse laser, or the like. The size of a laser spot is several hundreds μm. Laser is irradiated to the trabecular meshwork in the angle of the patient's eye.

The ophthalmic laser surgical apparatus according to the present disclosure is not limited to the above embodiment, but various modifications can be made thereto. The ophthalmic laser surgical apparatus according to the present disclosure includes also such modifications within a range where the technical idea is identical.

Moreover, in the present apparatus 500, information on the position of a laser spot (target) whose absolute position has been determined can be obtained, for example, by receiving reflected light at the laser spot position with a light receiving device and obtaining light intensity. Moreover, it may be configured to supply liquid only through the pipe 215.

It is sufficient if the alignment moving unit is configured to move at least in the Z direction. For example, the laser irradiation unit 100 and the eye fixation/interface unit 200 may be configured to be attached to another apparatus (coupled to the laser irradiation unit 100) such as a surgical microscope and configured for the operator to hold and move the XY alignment.

Moreover, the ophthalmic laser surgical apparatus according to the embodiment may be the following first to thirtieth ophthalmic laser surgical apparatuses. The first ophthalmic laser surgical apparatus is an ophthalmic laser surgical apparatus for operating a patient's eye with surgical laser, and includes a laser irradiation optical system being an irradiation optical system configured to irradiate a target position with the surgical laser and having a moving optical system configured to move a spot of the laser three-dimensionally, a fixation unit configured to fix the patient's eye on the laser irradiation optical system, an interface unit having an optical member configured to cover at least the cornea of the patient's eye, a tomography unit configured to photograph a tomographic image of the anterior segment of the patient's eye, and a monitoring unit configured to monitor the position of the fixation unit and/or the interface unit with respect to the patient's eye based on a tomographic image captured by the tomography unit.

In the first ophthalmic laser surgical apparatus, the second ophthalmic laser surgical apparatus has the tomography unit configured to include part of the interface unit in a tomographic image to be captured, and the monitoring unit having a monitor configured to display at least part of the fixation unit and/or the interface unit, the part having been captured by the tomography unit, to graphically indicate the position of the patient's eye and the fixation unit and/or the interface unit.

In the second ophthalmic laser surgical apparatus, the third ophthalmic laser surgical apparatus has the monitoring unit configured to detect position information of the interface unit based on the tomographic image, and the apparatus includes a display control unit configured to display on the monitor a symbol(s) indicating the fixation unit and/or the interface unit based on the detected position information of the interface unit.

In the third ophthalmic laser surgical apparatus, the fourth ophthalmic laser surgical apparatus includes a moving unit configured to move one of the fixation unit and the interface unit along a depth direction of the patient's eye, and has the monitoring unit configured to acquire position information of the fixation unit or the interface unit to be moved by the moving unit, and the display control unit configured to display on the monitor a symbol indicating the obtained fixation unit or interface unit.

In the fourth ophthalmic laser surgical apparatus, the fifth ophthalmic laser surgical apparatus has the symbol(s) of the fixation unit and/or the interface unit, the symbol(s) including at least a contact area of the fixation unit with the patient's eye or an area on the patient's eye side of the optical member.

In the third ophthalmic laser surgical apparatus, the sixth ophthalmic laser surgical apparatus includes an eye shape acquisition unit configured to acquire the shape of the patient's eye, and has the display control unit configured to display on the monitor a symbol indicating the patient's eye based on the shape of the eye obtained by the eye shape acquisition unit.

In the second ophthalmic laser surgical apparatus, the seventh ophthalmic laser surgical apparatus includes a display control unit configured to acquire the position of the corneal apex of the patient's eye or the position of the pupil center of the patient's eye from a tomographic image captured by the tomography unit and display a symbol indicating the position on the tomographic image.

In the second ophthalmic laser surgical apparatus, the eighth ophthalmic laser surgical apparatus includes a front image photographing unit configured to photograph a front image of the patient's eye, and a display control unit configured to acquire the position of the corneal apex of the patient's eye or the position of the pupil center of the patient's eye from a tomographic image captured by the tomography unit, and display a symbol indicating the obtained position of the corneal apex or the pupil center in a front image captured by the front image photographing unit.

In the first ophthalmic laser surgical apparatus, the ninth ophthalmic laser surgical apparatus includes a moving unit configured to move at least one of the fixation unit and the interface unit along a depth direction of the patient's eye, and has the monitoring unit including a control unit configured to control the moving unit based on the monitoring result.

In the ninth ophthalmic laser surgical apparatus, the tenth ophthalmic laser surgical apparatus has the tomography unit configured to show at least part of the interface unit in a tomographic image to be captured, and the monitoring unit configured to extract the position of the interface unit and the position of the patient's eye from a tomographic image captured by the tomography unit and control the moving unit based on the extraction result.

In the ninth ophthalmic laser surgical apparatus, the eleventh ophthalmic laser surgical apparatus has the monitoring unit including: a sensor configured to detect position information of the interface unit; and an eye position acquisition unit configured to acquire position information of the patient's eye based on a tomographic image captured by the tomography unit, and the control unit configured to control the moving unit based on the position information of the fixation unit or the interface unit, the position information having been detected by the sensor, and the position information obtained by the eye position acquisition unit.

In the ninth ophthalmic laser surgical apparatus, the twelfth ophthalmic laser surgical apparatus has the monitoring unit configured to acquire the position of the corneal apex of the patient's eye or the position of the pupil center of the patient's eye from a tomographic image captured by the tomography unit and obtain displacement information between the obtained position and a central axis of the fixation unit, the moving unit configured to be capable of further moving the fixation unit along a two-dimensional plane direction orthogonal to the depth direction of the patient's eye, and the control unit configured to control the moving unit based on the obtained displacement information, and cause the position of the corneal apex or the position of the pupil center to coincide with the central axis of the fixation unit or the interface unit.

In the eleventh ophthalmic laser surgical apparatus, the thirteenth ophthalmic laser surgical apparatus includes an eye shape acquisition unit configured to acquire the shape of the patient's eye, and has the eye position acquisition unit configured to acquire the shape of the eye obtained by the eye shape acquisition unit and position information of the patient's eye based on a tomographic image captured by the tomography unit.

In the first ophthalmic laser surgical apparatus, the fourteenth ophthalmic laser surgical apparatus includes an irradiation control unit configured to control the laser light source and the moving optical system and apply the surgical laser, and has the monitoring unit, further including an eye movement detecting unit configured to detect the movement of the patient's eye based on a tomographic image captured by the tomography unit, configured to stop the application of the laser to the patient's eye based on the detection result of the eye movement detecting unit.

In the first ophthalmic laser surgical apparatus, the fifteenth ophthalmic laser surgical apparatus has the monitoring unit configured to acquire axial information on an axis passing the corneal apex of the patient's eye or an axis passing the pupil center of the patient's eye based on a tomographic image captured by the tomography unit, acquire a direction of the patient's eye based on the obtained axial information, and obtain displacement information from a central axis of the fixation unit or the interface unit.

The sixteenth ophthalmic laser surgical apparatus includes a laser irradiation optical system configured to irradiate a patient's eye with surgical laser, a fixation unit configured to fix the patient's eye on the laser irradiation optical system, an interface unit having an optical member configured to cover the cornea of the patient's eye, a tomography unit configured to photograph a tomographic image of the anterior segment of the patient's eye, a monitor, and a control unit configured to display on the monitor the position of the fixation unit and/or the interface unit with respect to the patient's eye based on a tomographic image captured by the tomography unit.

In the sixteenth ophthalmic laser surgical apparatus, the seventeenth ophthalmic laser surgical apparatus has the tomography unit configured to include at least part of the fixation unit and/or the interface unit in a tomographic image to be captured, and the control unit configured to display on the monitor at least part of the fixation unit and/or the interface unit, the part having been captured by the tomography unit.

In the seventeenth ophthalmic laser surgical apparatus, the eighteenth ophthalmic laser surgical apparatus has the control unit configured to detect position information of the interface unit based on the tomographic image, and the control unit configured to display on the monitor a symbol(s) of the fixation unit and/or the interface unit based on the detected position information of the interface unit.

In the eighteenth ophthalmic laser surgical apparatus, the nineteenth ophthalmic laser surgical apparatus further includes a moving unit configured to move one of the fixation unit and the interface unit along a depth direction of the patient's eye, and has the control unit configured to detect position information of the fixation unit or the interface unit to be moved by the moving unit and display a symbol corresponding to the position information on the monitor.

In the nineteenth ophthalmic laser surgical apparatus, the twentieth ophthalmic laser surgical apparatus has the symbol indicating the fixation unit or the interface unit, the symbol indicating at least the position of a contact area of the fixation unit with the patient's eye or the position of an area on the patient's eye side of the optical member.

In the eighteenth ophthalmic laser surgical apparatus, the twenty-first ophthalmic laser surgical apparatus further includes an eye shape acquisition unit configured to acquire the shape of the patient's eye, and has the control unit configured to display a symbol indicating the patient's eye on the monitor based on the shape of the eye obtained by the eye shape acquisition unit.

In the seventeenth ophthalmic laser surgical apparatus, the twenty-second ophthalmic laser surgical apparatus is configured such that the control unit obtains the position of the corneal apex of the patient's eye or the position of the pupil center of the patient's eye from a tomographic image captured by the tomography unit, and displays a symbol indicating the obtained position on the tomographic image.

In the seventeenth ophthalmic laser surgical apparatus, the twenty-third ophthalmic laser surgical apparatus further includes a front image photographing unit configured to photograph a front image of the patient's eye, and has the control unit configured to acquire the position of the corneal apex of the patient's eye or the position of the pupil center of the patient's eye from a tomographic image captured by the tomography unit, and display a symbol indicating the obtained position of the corneal apex or the pupil center on a front image captured by the front image photographing unit.

In the sixteenth ophthalmic laser surgical apparatus, the twenty-fourth ophthalmic laser surgical apparatus further includes a moving unit configured to move at least one of the fixation unit and the interface unit along a depth direction of the patient's eye, and has the control unit configured to control the moving unit based on the position of the fixation unit and/or the interface unit with respect to the patient's eye.

In the twenty-fourth ophthalmic laser surgical apparatus, the twenty-fifth ophthalmic laser surgical apparatus has the tomography unit configured to show at least part of the interface unit in a tomographic image to be captured, and the control unit configured to extract the position of the interface unit and the position of the patient's eye from a tomographic image captured by the tomography unit and control the moving unit based on the extraction result.

In the twenty-fourth ophthalmic laser surgical apparatus, the twenty-sixth ophthalmic laser surgical apparatus further includes a sensor configured to detect position information of the fixation unit or the interface unit, and has the control unit configured to acquire position information of the patient's eye based on a tomographic image captured by the tomography unit and control the moving unit based on the position information of the fixation unit or the interface unit, the position information having been detected by the sensor, and the position information of the patient's eye.

In the twenty-fourth ophthalmic laser surgical apparatus, the twenty-seventh ophthalmic laser surgical apparatus has the moving unit configured to be capable of moving the fixation unit or the interface unit along a two-dimensional plane orthogonal to the depth direction of the patient's eye, and the control unit configured to acquire the position of the corneal apex of the patient's eye or the position of the pupil center of the patient's eye from a tomographic image captured by the tomography unit, further obtain displacement information indicating a deviation between the obtained position of the corneal apex or the pupil center and a central axis of the fixation unit or the interface unit, control the moving unit based on the displacement information, and cause the position of the corneal apex or the position of the pupil center to coincide with the central axis of the fixation unit or the interface unit.

In the twenty-sixth ophthalmic laser surgical apparatus, the twenty-eighth ophthalmic laser surgical apparatus is configured such that the control unit obtains the shape of the patient's eye, and obtains position information of the patient's eye based on the shape of the eye and a tomographic image captured by the tomography unit.

In the sixteenth ophthalmic laser surgical apparatus, the twenty-ninth ophthalmic laser surgical apparatus is configured such that the control unit detects the movement of the patient's eye based on a tomographic image captured by the tomography unit, and judges whether or not to stop the application of the laser to the patient's eye based on the detection result.

In the sixteenth ophthalmic laser surgical apparatus, the thirtieth ophthalmic laser surgical apparatus has the control unit configured to acquire axial information on an axis passing the corneal apex of the patient's eye or an axis passing the pupil center of the patient's eye based on a tomographic image captured by the tomography unit, acquire a direction of the patient's eye based on the obtained axial information, and obtain displacement information between the patient's eye and a central axis of the fixation unit or the interface unit based on the axial information and the direction of the patient's eye.

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. An ophthalmic laser surgical apparatus comprising:
a laser irradiation optical system configured to irradiate a patient's eye with surgical laser;
a fixation unit configured to fix the patient's eye on the laser irradiation optical system;
an interface unit including an optical member configured to cover at least the cornea of the patient's eye;
a tomography unit configured to capture a tomographic image of the anterior segment of the patient's eye; and
a monitoring unit configured to monitor a position of the fixation unit and/or the interface unit with respect to the patient's eye based on the tomographic image captured by the tomography unit.

2. The ophthalmic laser surgical apparatus according to claim 1, wherein
the tomography unit is configured to include at least part of the fixation unit and/or the interface unit in a tomographic image to be captured, and
the monitoring unit includes a monitor configured to display at least part of the fixation unit and/or the interface unit, the part having been captured by the tomography unit, to graphically indicate the position of the patient's eye and the position of the fixation unit and/or the interface unit.

3. The ophthalmic laser surgical apparatus according to claim 2, further comprising a display control unit configured to control display of the monitor.

4. The ophthalmic laser surgical apparatus according to claim 3, wherein
the monitoring unit is configured to detect position information of the fixation unit and/or the interface unit based on the tomographic image, and
the display control unit is configured to display on the monitor a symbol indicating the fixation unit based on the detected position information of the fixation unit and/or a symbol indicating the interface unit based on the interface unit.

5. The ophthalmic laser surgical apparatus according to claim 4, further comprising a moving unit configured to move one of the fixation unit and the interface unit along a depth direction of the patient's eye, wherein
the monitoring unit is configured to detect position information of the fixation unit or the interface unit to be moved by the moving unit, and
the display control unit displays on the monitor a symbol indicating the detected position of the fixation unit or the interface unit.

6. The ophthalmic laser surgical apparatus according to claim 5, wherein the symbol indicating the position of the fixation unit or the interface unit indicates at least a position of a contact area of the fixation unit with the patient's eye or a position of an area on the patient's eye side of the optical member.

7. The ophthalmic laser surgical apparatus according to claim 4, further comprising an eye shape acquisition unit configured to obtain a shape of the patient's eye, wherein the display control unit displays on the monitor a symbol indicating the patient's eye based on the shape of the eye obtained by the eye shape acquisition unit.

8. The ophthalmic laser surgical apparatus according to claim 3, wherein the display control unit is configured to obtain a position of the corneal apex of the patient's eye or a position of the pupil center of the patient's eye from the tomographic image captured by the tomography unit, and display a symbol indicating the obtained position on the tomographic image.

9. The ophthalmic laser surgical apparatus according to claim 3, further comprising a front image photographing unit configured to photograph a front image of the patient's eye, wherein the display control unit is configured to obtain a position of the corneal apex of the patient's eye or a position of the pupil center of the patient's eye from the tomographic image captured by the tomography unit, and display a symbol indicating the obtained position of the corneal apex or the pupil center on the front image captured by the front image photographing unit.

10. The ophthalmic laser surgical apparatus according to claim 1, further comprising a moving unit configured to move at least one of the fixation unit and the interface unit along a depth direction of the patient's eye, wherein the monitoring unit includes a control unit configured to control the moving unit based on a monitoring result.

11. The ophthalmic laser surgical apparatus according to claim 10, wherein the tomography unit is configured to show at least part of the interface unit in a tomographic image to be captured, and
the control unit extracts a position of the interface unit and a position of the patient's eye from the tomographic image captured by the tomography unit, and controls the moving unit based on the extraction result.

12. The ophthalmic laser surgical apparatus according to claim 10, wherein
the monitoring unit includes a sensor configured to detect position information of the fixation unit or the interface unit, and an eye position acquisition unit configured to obtain position information of the patient's eye based on the tomographic image captured by the tomography unit, and
the control unit controls the moving unit based on the position information of the fixation unit or the interface unit, the position information having been detected by the sensor, and the position information of the patient's eye, the position information having been obtained by the eye position acquisition unit.

13. The ophthalmic laser surgical apparatus according to claim 10, wherein
the monitoring unit is configured to obtain a position of the corneal apex of the patient's eye or a position of the pupil center of the patient's eye from the tomographic image captured by the tomography unit, and further obtain displacement information indicating a deviation between the obtained position of the corneal apex or the pupil center and a central axis of the fixation unit or the interface unit,
the moving unit permits the movement of the fixation unit or the interface unit along a two-dimensional plane orthogonal to the depth direction of the patient's eye, and
the control unit controls the moving unit based on the obtained displacement information, and causes the position of the corneal apex or the position of the pupil center to coincide with the central axis of the fixation unit or the interface unit.

14. The ophthalmic laser surgical apparatus according to claim 12, further comprising an eye shape acquisition unit configured to obtain a shape of the patient's eye, wherein the eye position acquisition unit is configured to obtain the position information of the patient's eye based on the shape of the eye obtained by the eye shape acquisition unit and the tomographic image captured by the tomography unit.

15. The ophthalmic laser surgical apparatus according to claim 1, wherein
the monitoring unit further includes an eye movement detecting unit configured to detect the movement of the patient's eye based on the tomographic image captured by the tomography unit, and
the monitoring unit is configured to stop application of the laser to the patient's eye based on the detection result of the eye movement detecting unit.

16. The ophthalmic laser surgical apparatus according to claim 1, wherein
the monitoring unit obtains:
axial information on an axis passing the corneal apex of the patient's eye or an axis passing the pupil center of the patient's eye based on a tomographic image captured by the tomography unit;
a direction of the patient's eye based on the obtained axial information; and
displacement information between the patient's eye and a central axis of the fixation unit or the interface unit based on the axial information and a direction of the patient's eye.
